(19) United States
(12) Patent Application Publication
(10) Pub. No.: US 2024/0099783 A1
(43) Pub. Date: Mar. 28, 2024

(54) BEACON DEVICES, SYSTEMS, AND METHODS FOR MEDICAL PROCEDURES

(71) Applicant: BOSTON SCIENTIFIC SCIMED, INC., Maple Grove, MN (US)

(72) Inventors: Gian Franco Loo Fuchs, San Jose (CR); Yeison Calvo, San Ramón (CR); Sean P. Fleury, Princeton, MA (US); Jose Roberto Soto Guzman, Moravia (CR)

(73) Assignee: BOSTON SCIENTIFIC SCIMED, INC., MAPLE GROVE, MN (US)

(21) Appl. No.: 18/373,706

(22) Filed: Sep. 27, 2023

Related U.S. Application Data
(60) Provisional application No. 63/410,679, filed on Sep. 28, 2022.

(51) Int. Cl. A61B 34/20

(52) U.S. Cl. CPC A61B 34/20; A61B 2034/2051; A61B 2034/2055; A61B 2034/2065

(57) ABSTRACT

A locator system having a flexible elongate delivery device, such as a flexible guidewire, advanced to a target site separately from the beacon element of the system. The beacon element may be configured to not extend beyond the perimeter of a housing on which the beacon element is mounted, thereby reducing the overall cross-sectional profile of the beacon portion of the system. The beacon element may be mounted with respect to the housing to be imaged in a direction transverse to the longitudinal axis of the system. The beacon element may include one or more LED's or other element imageable from a location separate from the target site.

### CROSS-REFERENCE TO RELATED APPLICATIONS

This application claims the benefit of priority under 35 U.S.C. § 119 to U.S. Provisional Application No. 63/410,679, filed Sep. 28, 2022, the entire disclosure of which is hereby incorporated by reference herein for all purposes.

### FIELD

The present disclosure relates generally to devices, systems, and methods used in performing procedures within a body, such as endoscopic procedures. More particularly, the present disclosure relates generally to devices, systems, and methods used to locate an anatomical site within a patient's body. Even more particularly, the present disclosure relates to devices, systems, and methods facilitating delivery of a locator device to an anatomical site within a patient's body.

### BACKGROUND

Viewing, locating, and manipulating anatomies, devices, and/or anatomies containing one or more devices from within a body may be difficult. During a procedure within the body, such as an endoscopic procedure within a body lumen (not involving cutting open the body), a medical professional may need to locate a particular anatomical structure of and/or position within the body. Locating a desired anatomical structure of and/or position within a body from another location within or outside the body may be difficult due to a lack of or low amount of illumination, and/or intervening anatomy, and/or the shape and/or configuration of various portions of the body. For instance, procedures within body lumens such as the intestines present lengthy regions of the anatomy which may be difficult to differentiate from outside the body. Various locating devices may be deployed within the body. However, because various passages within the body are tortuous, deploying locating devices transluminally through such tortuous passages may be challenging. For instance, pushability and trackability of a device through a tortuous body passage such as through the intestines can present challenges. The device may not advance properly and/or may loop back on itself and regress instead of advance. It is with respect to these and other considerations that the present disclosure may be useful.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a locator system includes a housing with a guide lumen extending therethrough configured to advance the housing over a flexible elongate delivery device to a target site within a patient's body; and a beacon element configured to be imaged by an imaging system remote from the target site.

In some embodiments, the housing has a wall defining a hollow interior within the housing; a window is defined through the housing wall; and the beacon element is positioned within the housing to be located through the window. In some embodiments, the beacon element emits a signal through the window radially outwardly with respect to the locator system. In some embodiments, a sleeve is positioned around the beacon element and the window to hold the beacon element within the housing, the sleeve allowing imaging of the beacon element therethrough. In some embodiments, a power lumen extends through the housing configured for passage of power connections therethrough from a power source to the beacon element.

In some embodiments, a beacon support is mounted on a distal end of the housing, and the beacon element is mounted on the beacon support. In some embodiments, the beacon element is mounted on the beacon support to be imageable from more than one direction radially with respect to the locator system. In some embodiments, the beacon support comprises a platform on which the beacon element is mounted.

In some embodiments, the housing has a cross-sectional area and the beacon is positioned within the cross-sectional area of the housing.

In some embodiments, the housing is formed as an extrusion,

In some embodiments, the beacon element is a light.

In accordance with various principles of the present disclosure, a locator system includes a housing with a guide lumen extending therethrough; a beacon element configured to be located by an imaging system remote from the target site; and a flexible elongate delivery device. In some aspects, the flexible elongate delivery device is slidably insertable into the guide lumen and the housing is slidable over the flexible elongate delivery device to be advanced independently of the flexible elongate delivery device to a target site within a patient's body

In some embodiments, the housing has a wall defining a hollow interior within the housing; a window is defined through the housing wall; and the beacon element is positioned within the housing to be imaged through the window.

In some embodiments, a beacon support is mounted on a distal end of the housing, and the beacon element is mounted on the beacon support.

In some embodiments, the housing has a cross-sectional area and the beacon is positioned within the cross-sectional area of the housing.

In accordance with various principles of the present disclosure, a method of locating a target site in a patient's body, from a location remote from the target site, includes advancing distal end of a flexible elongate delivery device to the target site; advancing a housing with a beacon element over the flexible elongate delivery device and to the distal end of the flexible elongate delivery device positioned at the target site; and imaging the beacon element from a location different from the target site.

In some aspects, the method further includes emitting a light signal from the beacon element radially with respect to the housing.

In some aspects, the method further includes emitting a light signal from the beacon element in more than one direction radially with respect to the housing.

In some aspects, the beacon element is positioned within the cross-sectional area of the housing, and the method further includes imaging the beacon element radially with respect to the housing.

In some embodiments, the method further includes imaging the distal end of the flexible elongate delivery device and the housing to advance the housing to the distal end of the flexible elongate delivery device at the target site.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of a locator system formed in accordance with various aspects of the present disclosure and positioned in a schematic representation of a gastrointestinal environment.

FIG. 2 illustrates a perspective view of an example of an embodiment of a locator system formed in accordance with various aspects of the present disclosure.

FIG. 3 illustrates a perspective view of a housing of an example of an embodiment of a locator system formed in accordance with various aspects of the present disclosure.

FIG. 4 illustrates a perspective view of a modified version of a locator system such as illustrated in FIG. 2.

FIG. 5 illustrates a perspective exploded view of another example of an embodiment of a locator system formed in accordance with various principles of the present disclosure.

FIG. 6 illustrates a perspective view of a locator system as in FIG. 5 assembled with a beacon element.

FIG. 7 illustrates an end view of a locator system as in FIG. 6.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. It will be appreciated that reference to a body passage includes naturally-existing passages (e.g., the pylorus) as well as medically-created passages (e.g., a passage created with the use of a medical instrument, such as between a stomach and jejunum) or otherwise. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, etc.) such location or site.

The present disclosure relates to devices, systems, and methods useful in performing a procedure, such as, without limitation, an endoscopic, laparoscopic, and/or open surgical procedure, within the body by initially identifying the target site for the procedure with a locator system, deploying the locator system at the target site, and then (e.g., at a later time) locating the locator system to perform the procedure at the desired target site identified by the locator system. A number of medical procedures require identification of the location of an anatomical structure, such as an organ or a tissue wall, and then delivering medical instruments to a second, different anatomical location to perform a procedure on or at the first-identified location. The first-identified location may be alternately referenced herein as a “target location”, a “target site”, a “treatment site”, a “deployment site”, a “target tissue”, a “target tissue site”, etc., without intent to limit. The second anatomical location or site may be referenced interchangeably herein as a different anatomical location, a separate anatomical location, an imaging site, a locating site, an access location, etc., without intent to limit. As may be appreciated, the terms site and location may be used interchangeably herein without intent to limit. Typically, principles of the present disclosure are applied with respect to a target location and a different anatomical location located internally within a patient. For instance, the target location and the imaging location may be separated by a tissue wall, and/or may be in different body organs or cavities. It will be appreciated that reference to “at” the target tissue site is intended to include tissue at and about the vicinity of (e.g., along, adjacent, etc.) the target tissue, and is not limited to just target tissue.

Non-limiting examples of procedures which may be performed with respect to a target location different from an imaging location include various medical procedures which involve moving a tissue wall (e.g., a body lumen wall or the wall of an organ) to a desired position, such as relative to another tissue wall (e.g., a body lumen wall or the wall of an organ). For instance, various procedures may be performed by entering the gastrointestinal (GI) tract through a first organ or structure (such as the esophagus, stomach, duodenum, small intestine, large intestine, or peritoneal cavity), and delivering an anchor or stent to adjacent organs or lumen or tissue structures (such as an adjacent portion of the GI tract, the bile duct, the pancreatic duct, the gallbladder, the pancreas, cysts, pseudocysts, abscesses, and the like). Typically, it is necessary to penetrate both an access tissue wall (e.g., a wall of a first organ or a first body lumen) at the access location, through which access is established to the target location, and a second tissue wall (e.g., of a wall of a second organ or a second body lumen) along or adjacent or at the target location at which the procedure is to be performed. A tissue anchor or stent or other device may be deployed between/across adjacent body lumens, organs, or other structures, such as to maintain tissue walls thereof in apposition, and/or to create an anastomosis, as indicated by the procedure. Tissue anchors may be used, in addition to the stent(s), to secure adjacent tissues or organs, such as before a stent is deployed, and may be left in place after the stent has been deployed.

Endoscopic procedures may be preferred over open surgical (involving cutting open the patient's body to gain access into the patient's body and internal anatomical structures) or laparoscopic (typically involving smaller incisions to gain access into the patient's body and internal anatomical structures) procedures for various reasons. For instance, open surgical or even laparoscopic procedures may involve longer recovery times than endoscopic methods which do not involve incisions, but which, instead, typically access the internal anatomy through a natural orifice and advancing equipment, tools, devices, etc., transluminally to the target area. However, endoscopic procedures present challenges with regard to locating a target location from a different anatomical location within the patient's body. In accordance with various principles of the present disclosure, a locator element and/or associated locator system is deployed at the target location to identify the target location from the different anatomical location

An example of a common endoscopic procedure presenting challenges in identifying a target location is a gastric bypass procedure in which a connection, such as an anastomosis, is created between the stomach and a certain part of the intestines through a gastroenteral anastomosis. For example, conditions of a disease may be benefitted by bypassing a portion of the duodenum, such as by creating an anastomosis, e.g., about 150 cm or greater from the pylorus, between the stomach and the small intestines (with a distal portion of the duodenum, or with the jejunum). In gastric outlet obstruction, a gastrojejunostomy serves the purpose of draining the contents of the stomach into the jejunum below/distal to the obstructed and/or dysfunctional duodenum. A gastrojejunostomy procedure may also serve as a minimally invasive and possibly reversible treatment option for patients with metabolic disease, by creating an anastomosis between the stomach and the jejunum to bypass the duodenum, with accompanying desired metabolic effects. In this manner, absorption of stomach contents (e.g., food and other nutrients) in the duodenum may be bypassed, and nutrients from such contents may not be absorbed, or uptake or absorption may be delayed, as such contents travel from the stomach through the small bowel, promoting patient weight loss and possibly controlling or resolving type-2 diabetes.

Endoscopic procedures, such as gastroenteral anastomoses, present various challenges, including the need to locate, endoscopically, a desired anatomical position (e.g., in the intestines) through a tissue wall, such as in a different anatomical location (e.g., via the gastric lumen). Ultrasound and/or fluoroscopy procedures provide images through anatomical walls (e.g., the gastric and enteral walls). However, although ultrasound is useful for imaging tissue, ultrasound may not image inorganic materials, used to identify tissue, as readily. Although fluoroscopy is well suited for viewing dense materials, such as those from which medical instruments are made, contrast mediums used with fluoroscopy may dissipate as the target tissue is located and may thus need to be reintroduced.

The use of a locator system at the target location may facilitate identification of the target location from a different anatomical location within the patient's body. Various locator systems include a locator element (e.g., a beacon such as a light or other signal generator or other element identifiable by imaging techniques). The locator element is inserted, such as with an endoscope, to the desired location for the procedure (e.g., where the connection of the intestines with the stomach is intended to be made). The endoscope is withdrawn, leaving the locator element in place. Then, the endoscope is advanced to the imaging site (optionally after being removed from the patient and the locator system, and then being re-inserted). One or more instruments for performing the procedure are inserted into and through the endoscope, such as to make an incision at the imaging site to access the target location (e.g., through the stomach wall and into the peritoneum). It will be appreciated that terms such as instruments, tools, devices, etc., may be used interchangeably herein without intent to limit. The locator element is visualized, or otherwise located, such as with the use of the endoscope, to identify the target site for the procedure, and the procedure may thus be performed at the desired target site. For instance, once in the stomach, the physician locates the desired section of the small intestine with the aid of a locator element within the small intestine (such as may be seen from outside the intestinal wall). The outside of the small intestine is accessed (e.g., by cutting through the stomach wall and extending a grasper through the peritoneum) and grasped and brought together with the stomach. A stent may then be deployed to connect the small intestine and the stomach, thereby creating the anastomosis.

Although locator systems may be delivered by an endoscope, in some instances an endoscope cannot readily reach the target location. For instance, the target location in a jejunum for a gastrojejunostomy may be too far for ready access by an endoscope. A locator system may be deployed from the distal end of the endoscope with the use of a guidewire. However, delivery of a locator system with a guidewire may present various challenges. For instance, a certain level of stiffness may be needed to support and/or to deliver the locator element. Additionally or alternatively, the thickness and/or stiffness of the resulting locator system and delivery system therefor (with the guidewire) may render the locator system difficult to maneuver through tortuous body passages (such as through the small intestines).

The present disclosure describes solutions to the above-discussed challenges of prior locator devices, systems, and method. In accordance with various principles of the present disclosure, locator devices, systems, and methods include a locator element deployed independently of a guidewire. In some aspects, the locator element is mounted on a housing capable of being guided over a flexible elongate element (e.g., a guidewire) which is delivered separately. More particularly, the housing may have a lumen through which a flexible elongate element may be passed so that the locator element may be guided over the flexible elongate element to the target location. As described above, and as may be appreciated by those of ordinary skill in the art, prior locator systems included guidewires on which a locator element is mounted. Such guidewires must be selected to have sufficient stiffness and thickness to adequately support and carry a locator element to a target location. By providing a locator element on a housing which may be guided over a separate flexible elongate element, a thinner more flexible elongate element may be used than may be used if the locator element were mounted on and delivered with the flexible elongate element. The thin, flexible elongate element may be readily navigated to the desired target location (with any known or heretofore known method), and the separate locator element may then be guided over the flexible elongate element to the target location. In some embodiments, the housing of the locator element is less flexible than the flexible elongate element so that the locator element may be advanced over the flexible elongate element to facilitate advancement of the locator element over the flexible elongate element. The housing may be an extrusion, such as a flexible tubular element, extendable over the flexible elongate element inserted separately for guiding the housing to the target site. In some embodiments, the proximal end of the housing extends proximally out of the patient, such as to control advancement of the housing and/or to hold the housing in place at the target site. In some embodiments, the housing does not extend so far as to extend proximally out of the patient. It will be appreciated that the term housing is used for the sake of convenience to refer to a housing or other type of element on which a locater element may be provided (in contrast with a flexible elongate element such as a guidewire), and without intent to limit to a particular longitudinal extent.

In some aspects, the locator element of a locator system formed in accordance with various principles of the present disclosure has a low profile with respect to the system to facilitate delivery to the target location. In some aspects, the position and orientation of the locator element on a locator system formed in accordance with various principles of the present disclosure allow increased range for the locator element to improve visualization and identification thereof by an imaging system at the imaging site.

It will be appreciated that locator devices, systems, and methods formed in accordance with various principles of the present disclosure may be used with various devices, systems, and methods for performing procedures within a patient's body other than those disclosed herein. Thus, although the devices and systems and methods described herein are described with respect to a gastrointestinal system, it may be understood that devices and systems and methods in accordance with the present disclosure may be advantageous for use in any other procedures. Moreover, it should be understood that the devices and systems and methods described herein may be used with other regions of the anatomy, such as anywhere selective location of tissue is through other tissue walls and/or is blind.

Various embodiments of locator devices, systems, and methods will now be described with reference to examples illustrated in the accompanying drawings. Reference in this specification to “one embodiment,” “an embodiment,” “some embodiments”, “other embodiments”, etc., indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of other embodiments as well. That is, any of the features, structures, concepts, and/or characteristics described herein can be mixed and matched to create hybrid embodiments, and such hybrid embodiment are within the scope of the present disclosure. Moreover, various features, structures, concepts, and/or characteristics of disclosed embodiments may be independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. Finally, it should be appreciated that various dimensions provided herein are examples, and one of ordinary skill in the art can readily determine the standard deviations and appropriate ranges of acceptable variations therefrom (e.g., in view of nominal dimensions, shape, stress/strain considerations, etc.) which are covered by the present disclosure and any claims associated therewith. The following description is of illustrative examples of embodiments only, and is not intended as limiting the broader aspects of the present disclosure.

It will be appreciated that common features in the drawings are identified by common reference elements and, for the sake of brevity and convenience, and without intent to limit, the descriptions of the common features are generally not repeated. For purposes of clarity, not all components having the same reference number are numbered. It will be appreciated that, in the following description, elements or components similar among the various illustrated embodiments with reference numbers greater than 100 are generally designated with the same reference numbers increased by a multiple of 100 and redundant description is generally omitted for the sake of brevity. Moreover, certain features in one embodiment may be used across different embodiments and are not necessarily individually labeled when appearing in different embodiments.

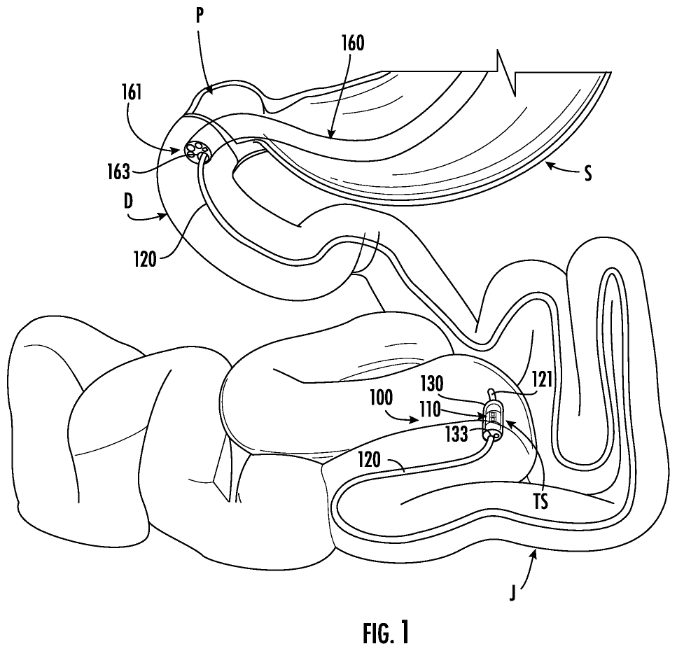

Turning now to the drawings, an example of an embodiment of an environment in which a locator system 100 formed in accordance with various principles of the present disclosure may be used is illustrated in FIG. 1. The locator system 100 includes a locator device 110 delivered with the assistance of a flexible elongate element 120 to a desired target site TS. The locator device 110 may be any device locatable by an imaging system or other locating system, such as a light (e.g., LED), and is referenced herein as a beacon device 110 for the sake of convenience and without intent to limit. In the example illustrated in FIG. 1, the beacon device 110 is delivered to a portion of a patient's jejunum J to be brought into apposition with a portion of the patient's stomach S. As may be appreciated, the flexible elongate element 120 must navigate through a tortuous pathway through various turns of the jejunum J.

In accordance with various principles of the present disclosure, to facilitate navigation of the flexible elongate element 120 to the target site TS, the flexible elongate element 120 is highly flexible elongate element such as a flexible guidewire such as known by those of ordinary skill in the art (e.g., a guidewire which may be used in endoscopic procedures, such as a guidewire with an outer diameter of under approximately 0.035″/0.889 mm). Moreover, in accordance with various principles of the present disclosure, the flexible elongate element 120 is deliverable to the target site TS independently of and prior to delivery of the beacon device 110. The flexible elongate element 120 may be referenced herein as a flexible elongate delivery device 120 such as to highlight the distinction of such element as delivered separately from the beacon device 110. As such, the flexible elongate delivery device 120 may navigate through tortuous pathways and flex as necessary without any impedance which may possibly be created by the provision of a beacon device 110 on the flexible elongate delivery device 120 during navigation to the target site TS.

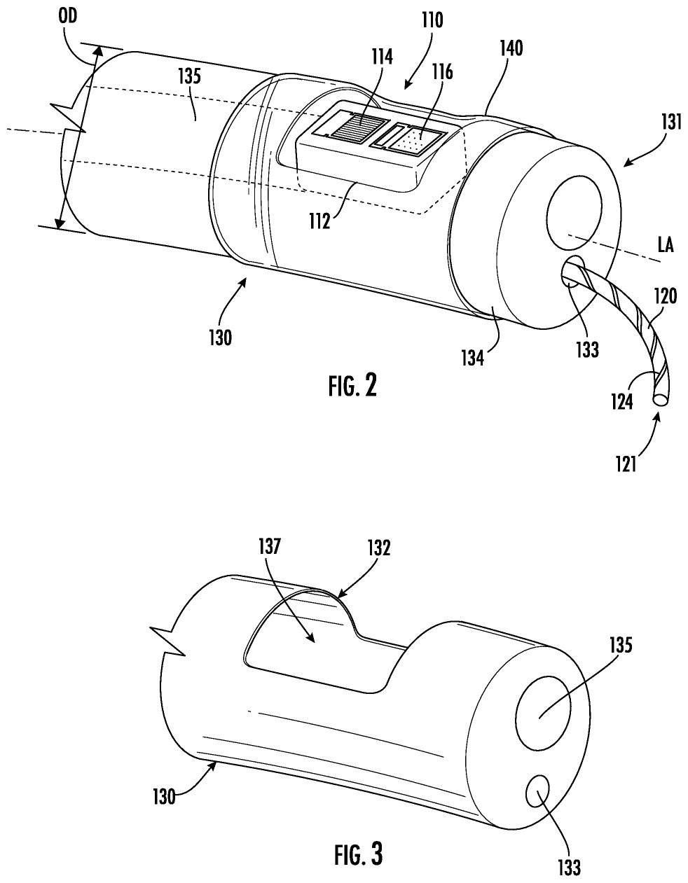

Further in accordance with various principles of the present disclosure, the beacon device 110 is mounted with respect to a housing 130 configured to be delivered to a target site TS with the assistance of the flexible elongate delivery device 120. More particularly, the housing 130 is configured to be delivered over the flexible elongate delivery device 120. Even more particularly, as may be appreciated with reference to FIG. 2, the housing 130 includes a guide lumen 133 therethrough configured to slidably receive the flexible elongate delivery device 120. The guide lumen 133 may extend generally along the longitudinal axis LA of the locator system 100. The flexible elongate delivery device 120 may be slidably inserted in the guide lumen 133 and the housing 130 may be advanced over the flexible elongate delivery device 120 distally into the patient until reaching the target site TS. As will be appreciated, initial deployment of the flexible elongate delivery device 120, and separate deployment of the housing 130 allows readier manipulation and navigation of the locator system 100 than achieved by previous systems. The beacon device 110 does not add to the stiffness of the flexible elongate delivery device 120 as in previous systems, so that the flexible elongate delivery device 120 may readily be navigated to the target site TS. The beacon device 110 is delivered proximal to the distal end 121 of the flexible elongate delivery device 120. The distal end 121 of the flexible elongate delivery device 120 may be delivered sufficiently past the target site TS to reduce the risk of the beacon device 110 being pushed past the distal end 121 of the flexible elongate delivery device 120 and off the flexible elongate delivery device 120. If the flexible elongate delivery device 120 is a guidewire, the distal end 121 may be even more flexible than proximal regions of the flexible elongate delivery device 120 and may include a radiopaque marker 124 by which advancement of the housing 130 over the flexible elongate delivery device 120 and to the distal end 121 thereof may be guided. In some embodiments, the locator system 100 may also include a radiopaque marker 134 position with respect to the beacon device 110 (e.g., on the housing 130 thereof) to facilitate positioning of the beacon device 110 with respect to the distal end 121 of the flexible elongate delivery device 120 and the target site TS, such as during the delivery of the beacon device 110, such as with the aid of fluoroscopy or other imaging systems.

The beacon device 110 may include a beacon housing 112 on which one or more beacon elements 114, 116 are mounted. In embodiments in which the beacon device 110 is powered externally (such as by being coupled, via wires, to an external power source outside the patient's body), such as illustrated in FIG. 2 and FIG. 3, the housing 130 also includes a power lumen 135 (e.g., extending generally along the longitudinal axis LA of the locator system 100) through which power connections (e.g., electrical wires) for powering the beacon device 110 may extend proximally to a power source (e.g., outside the patient's body).

Additional features of a locator system 100 formed in accordance with various principles of the present disclosure may be configured to improve navigation of the locator system 100 to the target site TS. For instance, in addition to increasing the flexibility of a locator system delivery device to improve navigation in tortuous body passages, a locator system 100 formed in accordance with various principles of the present disclosure may also be configured to minimize the cross-sectional area thereof and/or to improve smooth delivery to the target site TS. As may be appreciated with reference to the example of an embodiment of a locator system 100 illustrated in FIG. 2, and with reference to the example of an embodiment of a housing 130 of the locator system 100 illustrated in isolation in FIG. 3, the housing 130 includes a hollow interior 137 and a window 132 through a wall of the housing 130 adjacent the distal end 131 of the housing 130. The window 132 forms a lateral access to the hollow interior 137 of the housing 130 in which the beacon device 110 may be positioned. In the illustrated example of an embodiment, the beacon device 110 is mounted with respect to the housing 130 in a manner which allows visualization of the beacon generating portion of the beacon device 110 (e.g., an LED) to be visible through the window 132. Positioning of the beacon device 110 within the hollow interior 137 of the housing 130 allows the beacon device 110 to be mounted generally within the cross-sectional profile of the locator system housing 130 (not extending beyond the cross-sectional profile or perimeter of the housing 130), minimally impacting the cross-sectional dimensions of the locator system housing 130 if at all. Such configuration allows the overall cross-sectional profile or dimensions of the locator system 100 to be minimized compared with locator systems with beacons mounted over the housing or other delivery device thereof. For instance, the beacon device 110 does not project more than a few millimeters or a few fractions of a millimeter beyond the outer wall of the housing 130 such that the locator system 100 presents an atraumatic outer surface and navigation thereof is not impacted by the beacon device 110. In some embodiments, the outer diameter of the locator system 100 is selected based on the inner diameter of the working channel of an endoscope through which the locator system 100 is delivered. For instance, in some embodiments, the outer diameter of the locator system 100 is less than about 3.7 mm. In some aspects, a sleeve 140 is provided over the window 132 and the beacon device 110, such as to hold the beacon device 110 in place with respect to the window 132 and/or to present a smooth, atraumatic surface to tissue at the location of the beacon device 110 along the locator system 100. The sleeve 140 may be formed of a clear/semitransparent/transparent/translucent material allowing the beacon device 110 to be located therethrough (e.g., a light-transmitting material). Additionally or alternatively, the sleeve 140 may be formed of a flexible material, such as a heat shrinkable polymer, such as to facilitate positioning of the sleeve 140 over the beacon device 110 and housing 130.

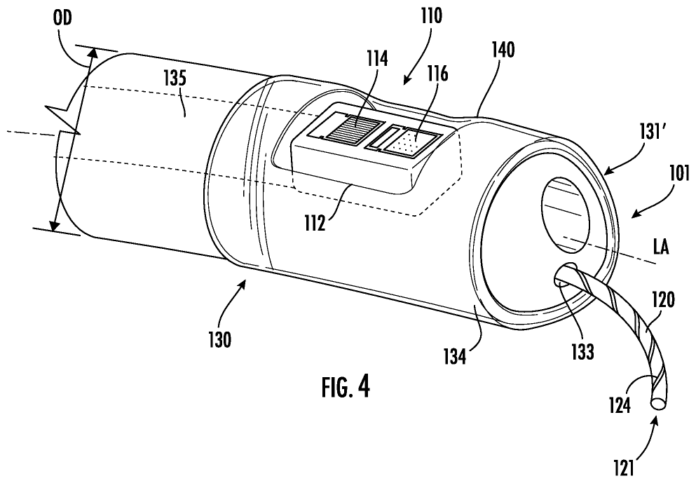

In the example of a modified embodiment of a locator system 100 illustrated in FIG. 4, sleeve 140 is extended over the distal end 131′ of the housing 130 and/or the distal end 131′ of the housing 130 itself is configured to form a blunt, atraumatic distal end 131′ of the locator system 100. Remaining elements of the locator system 100 illustrated in FIG. 4 are similar to elements of the locator system 100 illustrated in FIG. 2 and labeled with the same reference numerals, reference being made to the above descriptions thereof for the sake of brevity.

In accordance with various principles of the present disclosure, a beacon device 110 such as illustrated in FIG. 2 and FIG. 4 is positioned to emit a signal generally radially with respect to the locator system 100 and housing 130 thereof, and the flexible elongate delivery device 120. Such radial emission may facilitate pinpointing the location of the locator system 100 and the target site TS from the imaging site. In some embodiments, a locator system formed in accordance with various principles of the present disclosure may include more than one beacon element, such as to enhance locatability of the locator system 100 by an imaging system. For instance, the housing 130 of the locator system 100 illustrated in FIG. 2 or FIG. 3 may include more than one window 132 to allow more than one beacon device 110 to emit a signal in more than one direction radially with respect to the locator system 100, housing 130, and/or flexible elongate delivery device 120.

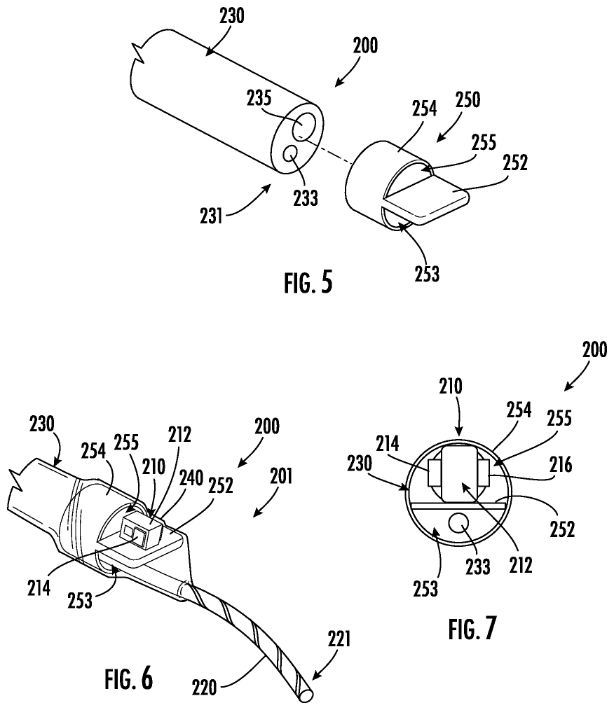

Various of the above-principles of the present disclosure may be implemented in other manners. For instance, another example of an embodiment of a locator system 200 with a beacon device 210 which may be delivered separately from a flexible elongate delivery device 220 is illustrated in FIG. 5, FIG. 6, and FIG. 7. Similar to the locator system 100 described above, the flexible elongate delivery device 220 of the locator system 200 illustrated in FIG. 5, FIGS. 6, and FIG. 7 is deliverable independently of and prior to delivery of the beacon device 210. As such, the flexible elongate delivery device 220 of the locator system 200 may be thinner than similar components of prior art locator systems which must be capable of supporting a beacon device or element thereon.

Also similar to the above-described locator system 100, the locator system 200 illustrated in FIG. 5, FIG. 6, and FIG. 7 is configured to be delivered separately from, such as over, a previously delivered flexible elongate delivery device 220 of the locator system 200. For instance, as may be seen in FIG. 5, the main housing 230 of the locator system 200 may be formed similar to the housing 130 of the above-described locator system 100, and include at least one guide lumen 233 through which the flexible elongate delivery device 220 may be passed. The main housing 230 may be advanced over the previously-delivered flexible elongate delivery device 220 (specifically, with the guide lumen 233 mounted over the flexible elongate delivery device 220) in a manner such as described above with respect to the locator system 100. In some aspects, the main housing 230 is a flexible tubular element. In some embodiments, such flexible tubular element has a proximal end extending proximally out of the patient, although other lengths of the main housing 230 are within the scope and spirit of the present disclosure.

Also similar to the locator system 200 described above, in accordance with various principles of the present disclosure, the beacon device 210 is mounted with respect to a housing 230 in a manner which minimizes the cross-sectional profile of the locator system 200. More particularly, as illustrated in FIG. 5, the locator system 200 includes a main housing 230 with a beacon device 210 supported/mounted on a beacon support 250 positioned at a distal end 231 of the main housing 230. The beacon support 250 provides a structure allowing the beacon device 210 to be mounted generally within the cross-sectional profile of the locator system housing 230, minimally impacting the cross-sectional dimensions of the locator system housing 230 if at all. In the example of an embodiment illustrated in FIG. 5, FIG. 6, and FIG. 7, the beacon support 250 includes a platform 252 on which the beacon device 210 is mounted, such as illustrated in FIG. 6 and FIG. 7. The platform 252 extends distally from the beacon support 250 and may be positioned to generally remain within the boundaries of (i.e., not projecting beyond) the cross-sectional profile of the main housing 230, with the beacon device 210 also remaining within the boundaries of the cross-sectional profile of the main housing 230 such as illustrated in FIG. 7. The platform 252 may have a rounded distal shape to form an atraumatic end which optionally facilitates navigation of the main housing 230 to a target site TS. As may be appreciated with reference to FIG. 5 and FIG. 7, the beacon support 250 includes a guide passage 253 in communication with the guide lumen 233 of the main housing 230 and through which the flexible elongate delivery device 220 may pass and by which the locator system 200 (particularly the beacon support 250 thereof) is mounted over the flexible elongate delivery device 220.

In some embodiments, the platform 252 may facilitate mounting of more than one beacon device 210 to allow the beacon device 210 to emit signals in more than one direction radially with respect to the locator system 200, such as may be appreciated with reference to FIG. 7. For instance, the beacon device 210 may have a beacon housing 212 with a beacon element 214, 216 mounted on each side thereof, such as illustrated in FIG. 6 and FIG. 7. In some embodiments, the beacon elements 214, 216 may be different in nature and positioned to emit signals in different (e.g., opposite) directions, or otherwise to be identified separately. Additionally, the beacon support 250 may include a power passage 255 allowing access to the power lumen 235 through the main housing 230 for access of power connections, such as wires, to the beacon device 210 through the main housing 230 and the beacon support 250. The platform 252 may be covered with a sleeve 240 (e.g., similar to the sleeve 140 described above) to hold the beacon device(s) 210 in place with respect to the beacon support 250. The beacon support 250 and the sleeve 240 may be configured to provide a generally blunt atraumatic shape to the distal end 201 of the locator system 200, such as illustrated in FIG. 6.

In some embodiments, the beacon support 250 is an end cap formed separately from and mounted on the main housing 230. The beacon support 250 may be secured to the distal end 231 of the main housing 230 such as by bonding (e.g., adhesive, solder, welding, etc.) or a mechanical securement (e.g., interference fit, friction fit, threaded engagement, etc.) or with a separate structural element such as the sleeve 140 described above or other coupling element such as a heat shrink element. In the illustrated example of an embodiment, the beacon support 250 includes a collar 254 configured to facilitate fitting of the beacon support 250 over the distal end 231 of the main housing 230.

Either or both of the housings 130, 230 of the above-described locator systems 100, 200 may be formed as extrusions, such as tubular extruded elements. The housings 130, 230 may be formed of any of a desired variety of polymers such as, without limitation, polyethylene, polyether block amides (e.g., Pebax©), polyimide, polypropylene and other common polymers used in medical device extrusions. The walls of the extrusion may be thin enough (depending on the material) to allow ready navigation through tortuous anatomical passages, yet sufficiently thick and/or stable to provide structural stability to the locator systems 100, 200. For instance, the walls may be at least about 0.003″ (0.0762 mm) thick. The housings 130, 230 may be extruded with the respective lumens 133, 135, 233, 235 formed by the extrusion process. It will be appreciated that although the proximal ends of the lumens 133, 135, 233, 235 are advantageously open to facilitate access thereto, the distal ends of the lumens 133, 135, 233, 235 may be closed in any a variety of manners known to those of ordinary skill in the art.

The beacon devices 110, 210 of the above-described locator systems 100, 200 may be any desired device or element capable of being identified and located by imaging system known or heretofore known to those of ordinary skill in the art. For instance, the beacon device 110, 210 may include one or more LED′S, such as arrays of LED's as described in U.S. Patent Application Publication US2021/0196106, titled Devices, Systems, And Methods For Locating A Body Lumen, published Jul. 1, 2021. For instance, in some embodiments, the LED's are different wavelengths (e.g., colors), such as to facilitate imaging from different distances and/or with different imaging equipment. Additionally or alternatively, the beacon device 110, 210 may be another types of signal-emitting or visualizable device or element known or heretofore known to those of ordinary skill in the art, the present disclosure not being limited in this regard.

It will be appreciated that other than some differences between the locator systems 100, 200 illustrated in FIGS. 1-7, the locator systems 100, 200 can be operated/used in substantially the same or similar manners. Accordingly, the common features have been identified by common reference elements and, for the sake of brevity, the descriptions of the common features have not been repeated. For purposes of clarity, not all components having the same reference number are numbered.

In use, the flexible elongate delivery device 120, 220 of a locator system 100, 200 formed in accordance with various principles of the present disclosure is advanced so that the distal end 121, 221 thereof is positioned at a target site TS within a patient. Typically, although not necessarily, the locator system 100, 200 is delivered through a working channel 163 of an endoscope 160, such as illustrated in FIG. 1. In the example of an embodiment of an environment illustrated in FIG. 1, the endoscope 160 is advanced through the patient's mouth, esophagus, and stomach (and optionally past the pylorus and into the duodenum). Because not all endoscopes are sufficiently long enough to extend to all target sites, the flexible elongate delivery device 120, 220 extends distally out of and beyond the distal end 161 of the endoscope 160 to the target site. For instance, as illustrated in FIG. 1, the distal end 161 of the endoscope 160 is extended through the stomach S and past the pylorus P, into the duodenum D, but may not extend much past the pylorus P. The flexible elongate delivery device 120, 220 is advanced distally from the endoscope 160, through the duodenum, and into the jejunum J of the patient. Once the distal end 121, 131 of the flexible elongate delivery device 120, 220 is advanced to and positioned at the target site TS, the flexible elongate delivery device 120, 220 is held in place with respect to the patient (e.g., by any manner known to those of ordinary skill in the art, such as by holding a proximal end of the flexible elongate delivery device 120, 220 in place, such as securing the proximal end to the patient or to stationary apparatus near the patient). The housing 130, 230 with the beacon device 110, 210 (and associated components) may then be advanced over the flexible elongate delivery device 120, 220. The outer dimensions of the housing 130, 230 (including the beacon device 110, 120) may be selected based on the inner diameter of the working channel 163 of the endoscope 160. For instance, as described above, the outer dimensions of the housing 130, 230 including the beacon device 110, 120 may be selected to fit within a working channel of a delivery device within which the locator system 100, 200 is delivered, such as less than the inner diameter of a typical endoscope (e.g., approximately 3.7 mm). The inner diameter of the power lumen 135, 235 (in which at least a portion of the beacon device 110, 210 may be positioned) may be approximately 0.12″±0.01″ (0.305 cm±0.02 cm). The inner diameter of the guide lumen 133, 233 within the housing 130, 230 may be selected based on the outer diameter of the flexible elongate delivery device 120, 220 to be slidably inserted therethrough. For instance, the inner diameter of the guide lumen 133, 233 may be approximately 0.027″±0.01″ (0.686 mm±0.2 mm). The housing 130, 230 is guided over the flexible elongate delivery device 120, 220, and advanced to the target site TS as well. The length of the housing 130, 230 may be selected to facilitate navigation through tortuous body passages. For instance, the length of the housing 130, 230 may be approximately 0.187″±0.05″ (0.475 cm±0.1 cm). If desired, the housing 130, 230 of the locator system 100, 200 is held in place with respect to the target site TS and the flexible elongate delivery device 120, 220 is withdrawn. For instance, if a proximal end of the housing 130, 230 extends proximally out of the patient's body, then such proximal end may be fixed, such as with respect to the patient or otherwise (e.g., in a manner similar to the manner in which the flexible elongate delivery device 120, 220 is held in place initially) to maintain the position of the beacon device 110, 210 with respect to the target site TS. An endoscope used to deliver the locator system 100, 200 may be withdrawn, and optionally then used to deliver an imaging system at another location within the patient to locate the locator system 100, 200. The beacon device 110, 210 is activated (or is already activated, if activation is even necessary) so that the locator system 100, 200 may be identified from another location within or outside the patient's body. An imaging system capable of identifying/locating the locator systems 100, 200 is then advanced to an anatomical location different from the target site TS to identify/locate the locator system 100, 200 (e.g., the beacon device 110, 120 thereof) at the target site TS. The endoscope 160 may be withdrawn, and instruments, tools, devices, etc. (such terms being used interchangeably herein without intent to limit) may then be advanced through the working channel 163 of the endoscope 160. Further steps, depending on the planned treatment/procedure to be performed at the target site TS, may then be performed. The endoscope 160 may be fully withdrawn off the flexible elongate delivery device 120, 220 so that the working channel 163 of the endoscope 160 provides a clear passage therein for other devices. In some embodiments, the locator system 100, 200 may be anchored with respect to the target site TS and the flexible elongate delivery device 120, 220 withdrawn so that there is no need to withdraw the endoscope 160 off the flexible elongate delivery device 120, 220. The endoscope 160 may include a visualization device capable of visualizing the locator system 100, 200, and/or additional imaging equipment may be used to locate the locator system 100, 200. For instance, the endoscope 160 may be positioned in the stomach S to locate the target site TS in the jejunum J so that an anastomosis may be formed between the stomach S and the jejunum J (e.g., to perform a gastric bypass). It will be appreciated that any component of the locator systems 100, 200 and/or the endoscope 160 may be controlled by a medical professional at a proximal end thereof in a manner known to those of ordinary skill in the art.

Although embodiments of the present disclosure may be described with specific reference to medical devices and systems and procedures for treating the gastrointestinal system, it should be appreciated that such medical devices and methods may be used to locate and treat tissues of the abdominal cavity, digestive system, urinary tract, reproductive tract, respiratory system, cardiovascular system, circulatory system, and the like. Although embodiments of the present disclosure may be described with specific reference to medical devices and systems (e.g., endoscopic devices, accessory tools, and/or guidewires) used in the GI system, it should be appreciated that locator devices and systems as described herein may be used with a variety of medical procedures performed in ductal, luminal, vascular, or body lumen anatomies, including, for example, interventional radiology procedures, balloon angioplasty/angiography procedures, thrombolysis procedures, urological or gynecological procedures, and the like. The medical devices herein may include a variety of medical devices for navigating body lumens, including, for example, catheters, ureteroscopes, bronchoscopes, colonoscopes, arthroscopes, cystoscopes, hysteroscopes, and the like. The disclosed medical devices and systems may also be inserted via different access points and approaches, e.g., percutaneously, endoscopically, laparoscopically, or combinations thereof.

In view of the above, it should be understood that the various embodiments illustrated in the figures have several separate and independent features, which each, at least alone, has unique benefits which are desirable for, yet not critical to, the presently disclosed locator devices, systems, and methods. Therefore, the various separate features described herein need not all be present in order to achieve at least some of the desired characteristics and/or benefits described herein. Only one of the various features may be present in a locator device, system, or method formed in accordance with various principles of the present disclosure. Alternatively, one or more of the features described with reference to one embodiment can be combined with one or more of the features of any of the other embodiments provided herein. That is, any of the features described herein can be mixed and matched to create hybrid designs, and such hybrid designs are within the scope of the present disclosure. Moreover, throughout the present disclosure, reference numbers are used to indicate a generic element or feature of the disclosed embodiment. The same reference number may be used to indicate elements or features that are not identical in form, shape, structure, etc., yet which provide similar functions or benefits. Additional reference characters (such as letters, as opposed to numbers) may be used to differentiate similar elements or features from one another.

It is to be understood by one of ordinary skill in the art that the present discussion is a description of illustrative examples of embodiments only, and is not intended as limiting the broader aspects of the present disclosure.

All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements, encompassing direct and indirect connections, unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A locator system comprising:
a housing with a guide lumen extending therethrough configured to advance said housing over a flexible elongate delivery device to a target site within a patient's body; and
a beacon element associated with said housing and configured to be imaged by an imaging system remote from the target site.

2. The locator system of claim 1, wherein:
said housing has a wall defining a hollow interior within said housing;
a window is defined through said housing wall; and
said beacon element is positioned within said housing to be located through the window.

3. The locator system of claim 2, wherein said beacon element emits a signal through the window radially outwardly with respect to said locator system.

4. The locator system of claim 2, further comprising a sleeve around said beacon element and said window to hold said beacon element within said housing, said sleeve allowing imaging of said beacon element therethrough.

5. The locator system of claim 2, wherein a power lumen extends through said housing configured for passage of power connections therethrough from a power source to said beacon element.

6. The locator system of claim 1, further comprising a beacon support mounted on a distal end of said housing, said beacon element mounted on said beacon support.

7. The locator system of claim 6, wherein said beacon element is mounted on said beacon support to be imageable from more than one direction radially with respect to said locator system.

8. The locator system of claim 6, wherein said beacon support comprises a platform on which said beacon element is mounted.

9. The locator system of claim 1, wherein said housing has a cross-sectional area and said beacon is positioned within the cross-sectional area of said housing.

10. The locator system of claim 1, wherein said housing is formed as an extrusion.

11. The locator system of claim 1, wherein said beacon element is a light.

12. A locator system comprising:
a housing with a guide lumen extending therethrough;
a beacon element associated with said housing and configured to be located by an imaging system remote from the target site; and
a flexible elongate delivery device;
wherein said flexible elongate delivery device is slidably insertable into the guide lumen and said housing is slidable over said flexible elongate delivery device to be advanced independently of said flexible elongate delivery device to a target site within a patient's body.

13. The locator system of claim 12, wherein:
said housing has a wall defining a hollow interior within said housing;
a window is defined through said housing wall; and
said beacon element is positioned within said housing to be imaged through the window.

14. The locator system of claim 12, further comprising a beacon support mounted on a distal end of said housing, said beacon element mounted on said beacon support.

15. The locator system of claim 12, wherein said housing has a cross-sectional area and said beacon is positioned within the cross-sectional area of said housing.

16. A method of locating a target site in a patient's body from a location remote from the target site, said method comprising:
advancing distal end of a flexible elongate delivery device to the target site;
advancing a housing with a beacon element over the flexible elongate delivery device and to the distal end of the flexible elongate delivery device positioned at the target site; and
imaging the beacon element from a location different from the target site.

17. The method of claim 16, further comprising emitting a light signal from the beacon element radially with respect to the housing.

18. The method of claim 17, further comprising emitting a light signal from the beacon element in more than one direction radially with respect to the housing.

19. The method of claim 16, wherein the beacon element is positioned within the cross-sectional area of the housing, said method further comprising imaging the beacon element radially with respect to the housing.

20. The method of claim 16, further comprising imaging the distal end of the flexible elongate delivery device and the housing to advance the housing to the distal end of the flexible elongate delivery device at the target site.
